(19) United States
(12) Patent Application Publication
(10) Pub. No.: US 2024/0008434 A1
(43) Pub. Date: Jan. 11, 2024

(54) Biofertilizer Endophytes of Cannabis

(71) Applicant: Agriculture Victoria Services PTY LTD, Bundoora, VICTORIA (AU)

(72) Inventors: Piyumi Niroshini Ekanayake, Doreen (AU); Dilani De Silva, South Morang (AU); Jatinder Kaur, Taylors Hill (AU); Tongda Li, Southbank (AU); Ross Mann, Coburg (AU); Timothy Ivor Sawbridge, Coburg (AU); German Carlos Spangenberg, Bundoora (AU)

(73) Assignee: Agriculture Victoria Services PTY LTD, Bundoora, VICTORIA (AU)

(21) Appl. No.: 18/039,658

(22) PCT Filed: Dec. 9, 2021
(86) PCT No.: PCT/AU2021/051473

§ 371 Date: May 31, 2023

(30) Foreign Application Priority Data
Dec. 10, 2020 (AU) 2020904593

(51) Int. Cl. A01H 3/00; A01H 17/00; C12N 1/20; A01H 6/28; A01H 5/06; A01N 63/20; A01P 21/00; C05F 11/08

(52) U.S. Cl. CPC A01H 3/00; A01H 17/00; C12N 1/205; A01H 6/28; A01H 5/06; A01N 63/20; A01P 21/00; C05F 11/08; C12R 2001/01

(57) ABSTRACT

The present invention relates to novel endophytes of plants of the Cannabaceae family, particularly biofertilizer Enterobacter sp. endophytes, and also to plants and parts thereof infected therewith, and related methods, including methods for conferring biofertilization to plants and for selecting a biofertilizer endophyte of a plant of the Cannabaceae family.

### FIELD OF THE INVENTION

The present invention relates to novel biofertilizer endophytes of plants of the Cannabaceae family, plants and parts infected therewith and related methods, including methods for conferring biofertilization to plants and for selecting a biofertilizer endophyte of a plant of the Cannabaceae family.

### BACKGROUND OF THE INVENTION

Efficient and consistent production of agricultural products in sufficient quantities, particularly those going to food and medicine, is a world-wide challenge. The field of terrestrial agriculture is relied upon to produce vast supplies of the world's food and medicinal products and textiles. Management of the economics, logistics and sheer scale of agricultural output is a considerable undertaking. However, consumers continue to demand higher quality products against the constant challenges faced by farmers in the production itself. These challenges include for example over cultivation of the same piece of land, due to the need to meet high demand for crops, which may lead to a decline in soil fertility and thus a reduction in productivity levels. While there is no one solution to address this issue, there are significant gains to be achieved from improving soil quality and/or agricultural productivity.

Cannabis has been used for its medicinal and psychoactive properties for centuries. Presently, it is believed that there are three distinct species in the genus: Cannabis sativa, Cannabis indica and Cannabis ruderalis. Cannabis and its extracts and derivatives such as hashish is a widely used drug around the world and is increasingly being recognised in the treatment of a range of medical conditions such as epilepsy, multiple sclerosis and chronic pain. While cannabis remains illicit in many countries, recent passed and proposed legislation seeks to legalise it for medical purposes, and in some countries even recreational use. Further, hemp forms of cannabis are also used to produce fibrous materials. In light of the increasing interest for cannabis use, there is inevitably a growing demand for its production, which creates a significant need to improve production of this crop.

While chemical fertilizers are widely used to combat the issue of soil fertility and/or agricultural productivity, this brings forth an associated set of drawbacks including, land and water contamination, and soil acidification amongst other detrimental effects to the environment as well as damage to the crops themselves.

Consequently, there exists a need to overcome, or at least alleviate, one or more of the difficulties or deficiencies associated with the prior art.

### SUMMARY OF THE INVENTION

In one aspect, the present invention provides a substantially purified or isolated Enterobacter sp. endophyte of a plant of the Cannabaceae family

By “endophyte” is meant an organism, generally a micro-organism that co-exists in a mutually beneficial relationship with a plant. Endophytes generally live on, in, or otherwise in close proximity to a plant and rely on the plant for survival, while at the same time confer a certain benefit to the plant. Endophytes of Enterobacter sp. are bacterial endophytes.

By “substantially purified” in the context of an endophyte is meant that the endophyte is free of other organisms. The term includes, for example, an endophyte in axenic culture. Preferably, the endophyte is at least approximately 90% pure, more preferably at least approximately 95% pure, even more preferably at least approximately 98% pure, even more preferably at least approximately 99% pure.

By “isolated” in the context of an endophyte is meant that the endophyte is removed from its original environment (e.g. the natural environment if it is naturally occurring; the plant). For example, a naturally occurring endophyte present in nature in a living plant is not isolated, but the same endophyte separated from some or all of the coexisting materials in the natural system, is isolated.

A plant of the Cannabaceae family includes plant parts thereof and may also be known as a cannabis plant or a hemp plant.

In a preferred embodiment, the plant of the Cannabaceae family from which a Enterobacter sp. endophyte is substantially purified or isolated is a Cannabis sativa, Cannabis indica or Cannabis ruderalis species plant, more preferably C. sativa. In particularly preferred embodiments, the plant of the Cannabaceae family is a C. sativa line denoted CannBio 2, CannBio 3, CannBio 4 and/or CannBio 5. A description of these plant lines is available in Australian Plant Breeder's Right (PBR) Application Nos 2017/253, 2017/254, 2017/255 and 2017/256, respectively.

The Enterobacter sp. endophyte may be substantially purified or isolated from any particular part of the plant, e.g. an organ. In preferred embodiments, the endophyte is substantially purified or isolated from a flower, flower bract, leaf, petiole, stem or root of the plant, more preferably a root.

The present invention arises from the discovery of Enterobacter sp. endophyte strains of plants of the Cannabaceae family and their ability to form mutually beneficial relationships with plants, that may be used to confer certain benefits to plants. In particular, the present invention arises further from the surprising discovery that said endophytes may be used by application to plants to confer a biofertilizer phenotype to a plant or part thereof, including for example nitrogen fixation.

Generally speaking, a biofertilizer endophyte possesses genetic and/or metabolic characteristics that result in a biofertilizer phenotype in a plant harbouring, or otherwise associated with, the endophyte. The biofertilizer phenotype may include nitrogen fixation in the plant with which the endophyte is associated, relative to a plant not associated with the endophyte, or instead associated with a control endophyte such as a Paenibacillus pabuli bacterial strain. The pests and/or diseases may include, but are not limited to, bacterial and/or fungal pathogens.

In a preferred embodiment, the endophyte is capable of conferring a biofertilizer phenotype to the plant or part thereof from which it is substantially purified or isolated and/or may be capable of conferring a biofertilizer phenotype to a plant or part thereof to which it is inoculated.

By “biofertilizer” phenotype is meant a preparation that contains live or dormant microorganisms, which when applied or inoculated into a plant or plant part, assists said plant or plant part, with accessing nutrient availability in the soil or other growth support medium, thus improving fertility and productivity of plant and soil.

In another preferred embodiment of this aspect of the present invention, the biofertilizer phenotype may be the enhanced growth of the plant under conditions of below normal, or substantially absent, nitrogen levels as compared with a plant that is absent of the endophyte. More preferably, the biofertilizer phenotype may be associated with changes to expression of a nitrogen fixation (nif) gene cluster, which may include, but is not limited to nifA, nifB, nifD, nifF, nifH, nifJ, nifK, nifL, nifS, and nifW. Even more preferably, the biofertilizer phenotype may be associated with up-regulation of nifA, nifB, nifF and nif, and/or the downregulation of nifD, nifH, nifJ and nifK. Most preferably, the biofertilizer phenotype may be associated with enhanced growth of a plant or part thereof, wherein the plant part is a root or a shoot. In a preferred embodiment, enhanced growth of the root length may be between about 2%-30% longer relative to an uninoculated control plant and/or have enhanced growth of the shoot length of between about 2%-26% longer relative to an uninoculated control plant, after a suitable period of time, for example at least 7 days after inoculation

By “inoculated” is meant to be placed in association with a plant to form a mutually beneficial relationship with a plant, whether that be on, in, or otherwise in close proximity to the plant. In preferred embodiments, the plant or part thereof to which the endophyte is inoculated is first free of that endophyte. In preferred embodiments, the inoculation method may be by soaking seeds in bacterial culture.

In another preferred embodiment of this aspect of the present invention, the endophyte may be isolated from roots of the plant.

In preferred embodiments, the Enterobacter sp. endophyte may be an Enterobacter sp. strain, preferably a strain denoted EB-008, EB-016, and/or EB-018, as deposited with the National Measurement Institute of 1/153 Bertie St, Port Melbourne, Victoria 3207 Australia on 24 Nov. 2020 with accession numbers V20/025721, V20/025724, and V20/025726, respectively.

Accordingly, in another aspect, the present invention provides a substantially purified or isolated biofertilizer endophyte selected from the group consisting of EB-008, EB-016, and EB-018 as deposited with The National Measurement Institute of 1/153 Bertie St, Port Melbourne, Victoria 3207 Australia on 24 Nov. 2020 with accession numbers V20/025721, V20/025724, and V20/025726, respectively.

All endophyte strains denoted EB-008, EB-016, and EB-018 were substantially purified or isolated from the roots of a CannBio line of a C. sativa plant. All named endophytes are biofertilizer endophytes with the ability to confer one or more biofertilizer properties to a plant—at least a plant of the Cannabaceae family (e.g. C. sativa).

In a further aspect of the present invention there is provided a plant or part thereof inoculated with one or more Enterobacter sp. endophytes as hereinbefore described.

In a preferred embodiment of this aspect of the present invention, the endophyte may confer a biofertilizer phenotype to a plant or part thereof.

In another preferred embodiment of this aspect of the present invention, the biofertilizer phenotype may be the enhanced growth of the plant under conditions of below normal, or substantially absent, nitrogen levels as compared to a plant that is absent of the endophyte. More preferably, the biofertilizer phenotype may be associated with enhanced growth of a plant or part thereof, wherein the plant part may be a root or a shoot. In a preferred embodiment, enhanced growth of the root length may be between about 2%-30% longer relative to an uninoculated control plant and/or have enhanced growth of the shoot length of between about 2%-26% longer relative to an uninoculated control plant, after a suitable period of time, for example at least 7 days after inoculation. More preferably, the plant of the Cannabaceae family may be a Cannabis sativa species plant.

The endophytes of the present invention may have the ability to be transferred through propagative material from one plant generation to the next. The endophyte may then spread or locate to other tissues as the plant grows, i.e. to roots. Alternatively, or in addition, the endophyte may be recruited to the plant root, e.g. from soil, and spread or locate to other tissues. In either sense, the endophyte may be said to be stably inoculated/infected to the plant.

Therefore, the present invention also provides a plant, plant propagative material or other plant part derived from a plant inoculated with an endophyte as herein described and infected therewith.

The present invention provides the use of an endophyte as herein described to produce a plant or part thereof infected, preferably stably infected, with said one or more of said endophytes.

The present invention also provides a method for conferring a biofertilizer phenotype to a plant or part thereof, said method including inoculating to the plant or part thereof an endophyte as herein described. In all preferred embodiments, the plant or plant part inoculated or otherwise infected with an endophyte as herein described will exhibit an endophyte-conferred biofertilizer phenotype, or in other words, the endophyte will confer thereto a biofertilizer endophyte.

In a preferred embodiment of this aspect of the present invention, the plant or part thereof may be free of said endophyte prior to inoculation and may be stably infected with said endophyte.

In a preferred embodiment the method for conferring a biofertilizer phenotype to a plant or part thereof includes the steps of:
a. substantially purifying or isolating one or more endophytes; and
b. inoculating the plant or part thereof with the isolated endophyte(s).

In a preferred embodiment, the method for conferring a biofertilizer phenotype to a plant or part thereof includes subjecting said one or more isolated endophytes to microbiome profiling, analysing the transcriptome of said one or more endophytes, preferably via sequencing, to identify expression of one or more genes associated with nitrogen fixation, and
selecting one or more endophytes which are capable of conferring a biofertilizer phenotype to the plant from which it is substantially purified or isolated and/or is capable of conferring a biofertilizer phenotype to a plant or part thereof to which it is inoculated, wherein the plant or part thereof is inoculated with the selected endophyte.

Plants are often associated with many endophyte species and strains with varying functions and properties. The present invention also provides an efficient method for selecting in particular a biofertilizer endophyte of a plant of the Cannabaceae family.

Thus, in another aspect, the present invention provides a method for selecting a biofertilizer endophyte of a plant of the Cannabaceae family, said method comprising:
a. substantially purifying or isolating one or more endophytes,
b. subjecting said one or more endophytes to microbiome profiling,
c. analysing the transcriptome of said one or more endophytes, preferably via sequencing, to identify expression of one or more genes associated with nitrogen fixation, and
d. selecting an endophyte which may be capable of conferring a biofertilizer phenotype to the plant from which it is substantially purified or isolated and/or may be capable of conferring a biofertilizer phenotype to a plant or part thereof to which it is inoculated.

In a preferred embodiment of this aspect of the present invention, the step of substantially purifying or isolating one or more endophytes may include providing one or more samples of said plant or part thereof, preparing an extract(s) from said sample(s), and growing bacterial colonies from said extract(s).

In a preferred embodiment, the sample of plant material may be selected from one or more of the group consisting of flowers, flower bracts, leaves, petioles, roots and stem

In a preferred embodiment, the one or more genes associated with nitrogen fixation may be a nitrogen fixation (nif) gene cluster, which may include, but is not limited to nifA, nifB, nifD, nifF, nifH, nifJ, nifK, nifL, nifS, and nifW.

In another preferred embodiment of this aspect of the present invention, the step of subjecting the endophyte to microbiome profiling may include generating sequence data by metagenomics sequencing. More preferably, metagenomics sequencing may be conducted using primers directed to a 16S rRNA gene and PNA PCR blockers. Even more preferably, the primers may be directed to a V4 region of the 16S rRNA gene. Most preferably, the V4 region may include 515F and 806R of the 16S rRNA gene.

In a preferred embodiment of this aspect of the present invention, the biofertilizer phenotype may be enhanced growth of the plant under conditions of below normal, or substantially absent, nitrogen levels as compared to a plant that may be absent of the endophyte. More preferably, the biofertilizer phenotype may be associated with changes to expression of a nif gene cluster, which may include, but is not limited to nifA, nifB, nifD, nifF, nifH, nifJ, nifK, nifL, nifS, and nifW. Even more preferably, the biofertilizer phenotype may be associated with up-regulation of nifA, nifB, nifF and nifL, and/or the downregulation of nifD, nifH, nifJ and nifK.

In a particularly preferred embodiment, the nifA gene may include a nucleotide sequence selected from the group consisting of:
(a) the sequences shown in SEQ ID NOS: 4 and 5; and
(b) functionally active variants of the sequences recited in (a).

In a particularly preferred embodiment, the nifB gene may include a nucleotide sequence selected from the group consisting of:
(a) the sequence shown in SEQ ID NO: 6; and
(b) functionally active variants of the sequence recited in (a).

In a particularly preferred embodiment, the nifD gene may include a nucleotide sequence selected from the group consisting of:
(a) the sequence shown in SEQ ID NO: 7; and
(b) functionally active variants of the sequence recited in (a).

In a particularly preferred embodiment, the nifF gene may include a nucleotide sequence selected from the group consisting of:
(a) the sequence shown in SEQ ID NO: 8; and
(b) functionally active variants of the sequence recited in (a).

In a particularly preferred embodiment, the nifH gene may include a nucleotide sequence selected from the group consisting of:
(a) the sequences shown in SEQ ID NOs: 9 and 10; and
(b) functionally active variants of the sequences recited in (a).

In a particularly preferred embodiment, the nifJ gene may include a nucleotide sequence selected from the group consisting of:
(a) the sequences shown in SEQ ID NOs: 11 and 12; and
(b) functionally active variants of the sequences recited in (a).

In a particularly preferred embodiment, the nifK gene may include a nucleotide sequence selected from the group consisting of:
(a) the sequence shown in SEQ ID NO: 13; and
(b) functionally active variants of the sequences recited in (a).

In a particularly preferred embodiment, the nifL gene may include a nucleotide sequence selected from the group consisting of:
(a) the sequence shown in SEQ ID NO: 14; and
(b) functionally active variants of the sequences recited in (a).

In a particularly preferred embodiment, the nifS gene may include a nucleotide sequence selected from the group consisting of:
(a) the sequence shown in SEQ ID NO: 15; and
(b) functionally active variants of the sequences recited in (a).

In a particularly preferred embodiment, the nifW gene may include a nucleotide sequence selected from the group consisting of:
(a) the sequence shown in SEQ ID NO: 16; and
(b) functionally active variants of the sequences recited in (a).

The present invention encompasses changes to expression of a functionally active variant (such as an analogue, derivative or mutant) of the specified nif gene. By ‘functionally active’ in relation to the nif gene is meant that changes to the expression of the variant is capable of conferring a biofertilizer phenotype on a plant.

Such variants include naturally occurring allelic variants and non-naturally occurring variants. Additions, deletions, substitutions and derivatizations of one or more of the nucleotides are contemplated so long as the modifications do not result in loss of functional activity of the variant. Preferably the functionally active variant has at least approximately 90% identity to the specified sequence to which the variant corresponds, more preferably at least approximately 95% identity, even more preferably at least approximately 98% identity, most preferably at least approximately 99% identity. Such functionally active variants include, for example, those having conservative nucleic acid changes.

By ‘conservative nucleic acid changes’ is meant nucleic acid substitutions that result in conservation of the amino acid in the encoded protein, due to the degeneracy of the genetic code. Such functionally active variants also include, for example, those having nucleic acid changes which result in conservative amino acid substitutions of one or more residues in the corresponding amino acid sequence.

By ‘conservative amino acid substitutions’ is meant the substitution of an amino acid by another one of the same class, the classes being as follows:
Nonpolar: Ala, Val, Leu, Ile, Pro, Met Phe, Trp
Uncharged polar: Gly, Ser, Thr, Cys, Tyr, Asn, GIn
Acidic: Asp, Glu
Basic: Lys, Arg, His
   Other conservative amino acid substitutions may also be made as follows:
Aromatic: Phe, Tyr, His
Proton Donor: Asn, GIn, Lys, Arg, His, Trp
Proton Acceptor: Glu, Asp, Thr, Ser, Tyr, Asn, GIn

In this specification, the term ‘comprises’ and its variants are not intended to exclude the presence of other integers, components or steps.

In this specification, reference to any prior art in the specification is not and should not be taken as an acknowledgement or any form of suggestion that this prior art forms part of the common general knowledge in Australia or any other jurisdiction or that this prior art could reasonably expected to be combined by a person skilled in the art.

The present invention will now be more fully described with reference to the accompanying Examples and drawings. It should be understood, however, that the description following is illustrative only and should not be taken in any way as a restriction on the generality of the invention described above.

### BRIEF DESCRIPTION OF THE FIGURES

FIG. 1 represents an Average Nucleotide Identity (ANI) analysis of novel bacterial strains EB-008, EB-016 and EB-018 with genome sequences of E. soli and E. asburiae isolates from NCBI.

FIG. 2 represents a pan-genome (Roary) of novel bacterial strains EB-008, EB-016 and EB-018 with genome sequences of E. soli and E. asburiae isolates from NCBI.

FIG. 3 represents nif gene clusters in the novel bacterial strains EB-018, EB-008, EB-016 identified using BLASTn.

FIG. 4 represents transcriptomic analysis of the expression of the nif gene clusters in two novel bacterial strains (EB-016 and EB-018).

FIG. 5 represents the average shoot and root length of tomato seedlings inoculated with novel bacterial strains (EB-008, EB-016, EB-018) and E. coli after 10 days growth on Burks medium (no nitrogen) and MS medium (normal nitrogen).

FIG. 6 represents the distribution of novel bacterial strain EB-016 throughout a mature medicinal cannabis plant (F—Flower, FL—Flower Bract, O-L—Old Leaf, Y-L—Young Leaf, O-P—Old Petiole, Y-P—Young Petiole, STEM—Stem, R—Root). The microbiome profile was established by mapping 16S metagenomic reads from the above organs to the 16S sequence of EB-016.

### DETAILED DESCRIPTION OF EMBODIMENTS

In the following examples it is demonstrated that three novel plant associated Enterobacter sp. bacterial strains EB-008, EB-016 and EB-018 were isolated from medicinal cannabis (Cannabis sativa) plants. Each of the novel strains display the ability to increase the growth plants when grown without nitrogen and with normal nitrogen levels. The genomes of the three novel bacterial strains have been sequenced and represent a novel Enterobacter species. Analysis of the genome sequences showed all three bacterial strains have a nitrogen fixation gene cluster, which was transcriptionally active when strains were grown without nitrogen and with normal nitrogen levels. The three novel strains were ubiquitously distributed throughout organs of medicinal cannabis but was most concentrated in the roots.

### Example 1—Isolation of Bacterial Strains

Leaves, petioles, stems, flowers and roots were harvested from four different chemotypes (lines) (Cannbio—2, 3, 4, 5) of mature cannabis plants. Plants were grown in a greenhouse in pots containing two different substrates: standard potting mix and coconut matting/Jiffy. Root tissues were washed in sterile distilled water to remove soil particles and all the harvested tissues were cut into approximately 1 cm² pieces. The plant tissues and organs belonging to different Cannbio lines were separately placed in micro collection tubes and submerged in sufficient Phosphate Buffered Saline (PBS) to completely cover the plant tissue. Plant tissues were ground using a Qiagen TissueLyser II, for 1 minute at 30 Hertz. A 10 μl aliquot of the macerate was added to 90 μl of PBS. Subsequent 1 in 10 dilutions of the 10⁻¹ suspension were used to create additional 10⁻² to 10⁻⁴ suspensions. Once the suspensions were well mixed, 50 μl aliquots of each suspension were plated onto Reasoners 2 Agar (R2A) for growth of bacteria. Dilutions that provided a good separation of bacterial colonies were subsequently used for isolation of individual bacterial colonies through re-streaking of single bacterial colonies from the dilution plates onto single R2A plates to establish a pure bacterial colony.

Around 126 bacterial strains were obtained from mature plants grown in standard potting mix.

The novel bacterial strains EB-008 and EB-018 were collected from roots of medicinal cannabis plants Cannbio 59 and EB016 from Cannbio 3.

### Example 2—Identification of Novel Bacterial Strains

### Genomics

The genomes of novel bacterial strains EB-008, EB-016 and EB-018 were sequenced. These novel bacterial strains were retrieved from the glycerol collection stored at −80° C. by streaking on NA plates. Single colonies from these plates were grown overnight in Nutrient Broth and pelleted. These pellets were used for genomic DNA extraction using the bacteria protocol of Wizard® Genomic DNA Purification Kit (A1120, Promega). DNA sequencing libraries were generated for Illumina sequencing using the Illumina Nextera XT DNA library prep protocol. All libraries were sequenced using an Illumina HiSeq platform. Raw reads from the sequencer were filtered to remove any adapter and index sequences as well as low quality bases using fastp (Chen, Zhou, et al., 2018) quality controller software for fastq files. To enable full genome assembly, long reads were generated for the three novel bacterial strains by sequencing DNA using Oxford Nanopore Technologies (ONT) MinION platform. The DNA from the Wizard® Genomic DNA Purification Kit was first assessed with the genomic assay on Agilent 2200 TapeStation system (Agilent Technologies, Santa Clara, CA, USA) for integrity (average molecular weight 30 Kb). The sequencing library was prepared using an in-house protocol modified from the official protocols for transposases-based library preparation kits (SQK-RAD004/SQK-RBK004, ONT, Oxford, UK). All libraries were sequenced on a MinION Mk1 B platform (MIN-101B) with R9.4 flow cells (FLO-MIN106) and under the control of MinKNOW software. After the sequencing run finished, the fast5 files that contain raw read signals were transferred to a separate, high performance computing Linux server for local basecalling using ONT's Albacore software (Version 2.3.1) with default parameters. For libraries prepared with the barcoding kit (SQK-RBK004), barcode demultiplexing was achieved during basecalling. The sequencing summary file produced by Albacore was processed by the R script minion qc (https://github.com/roblanf/minion_qc) and NanoPlot (De Coster et al. 2018) to assess the quality of each sequencing run, while Porechop (Version 0.2.3, https://github.com/rrwick/Porechop) was used to remove adapter sequences from the reads. Reads which were shorter than 300 bp were removed and the worst 5% of reads (based on quality) were discarded by using Filtlong (Version 0.2.0, https://github.com/rrwick/Filtlong).

The whole genome sequence of the three novel bacterial strains were assembled using Unicycler (Wick et al. 2017). Unicycler performed hybrid assembly when both Illumina reads, and MinION reads were available. MinION reads were mainly used to resolve repeat regions in the genome, whereas Illumina reads were used by Pilon (Walker et al. 2014) to correct small base-level errors. Multiple rounds of Racon (Vaser et al. 2017) polishing were then carried out to generate consensus sequences. Assembly graphs were visualised by using Bandage (Wick et al. 2015).

A complete circular chromosome sequence was produced for the three novel bacterial strains. The genome size for the novel bacterial strains EB-008, EB-016 and EB-018 were 4,929,453 bp, 5,017,261 bp and 5,112,947 bp respectively (Table 1).

TABLE 1
Summary of properties of the final genome sequence assembly

| Strain | Genome size | GC content | Coverage | Coverage |
|---|---|---|---|---|
| ID | (bp) | (%) | Illumina reads | ONT MinION |
| EB-008 | 4,929,453 | 53.2 | 160.6x | 461.9x |
| EB-016 | 5,017,261 | 53.2 | 65.5x | 645.6x |
| EB-018 | 5,112,947 | 52.9 | 92.3x | 105.1x |

The percent GC content ranged from 52%-53%. These novel bacterial strains were annotated by Prokka (Seemann 2014) with a custom, genus-specific protein database to predict genes and corresponding functions, which were then screened manually to identify specific traits.

The number of genes for the novel bacterial strains EB-008, EB-016 and EB-018 were 6,268, 6,562 and 4,828 genes respectively (Table 2).

TABLE 2
Summary of genome coding regions

| | No. of | No. of | No. of | No. of | No. of |
|---|---|---|---|---|---|
| Strain ID | tRNA | tmRNA | rRNA | CDS | gene |
| EB-008 | 84 | 1 | 22 | 6161 | 6268 |
| EB-016 | 83 | 1 | 22 | 6456 | 6562 |
| EB-018 | 83 | 1 | 22 | 4722 | 4828 |

A phylogenetic analysis of the novel bacterial strains EB-008, EB-016 and EB-018 was undertaken by sequence homology comparison of the 16S rRNA gene regions extracted from whole genome sequence of each bacteria (Table 3, SEQ ID NOs: 1, 2 and 3).

TABLE 3
16s rRNA gene regions identified within Enterobacter
strains EB-008, EB-016 and EB-018

| | | Nucleic acid |
|---|---|---|
| Enterobacter strain | Gene | SEQ ID NO: |
| EB008 | 16s rRNA | 1 |
| EB016 | 16s rRNA | 2 |
| EB018 | 16s rRNA | 3 |

The sequences were aligned by BLASTn on NCBI against the non-redundant nucleotide database and the 16S ribosomal RNA database. The preliminary taxonomic identification of the novel bacterial strains EB-008, EB-016 and EB-018 were Enterobacter sp. closely related to E. soli (Tables 4 and 5).

TABLE 4
BLASTn hit against database nr; Enterobacter sp. strain
LSRC69 16S ribosomal RNA gene, partial sequence

| | Query | E- | % | | |
|---|---|---|---|---|---|
| | Coverage | Value | Identity | Species | Accession |
| EB-008 | 100% | 0 | 99.58% | Enterobacter sp. | JF772075.1 |
| EB-016 | 100% | 0 | 99.72% | Enterobacter sp. | JF772075.1 |
| EB-018 | 100% | 0 | 99.27% | Enterobacter sp. | JF772075.1 |

TABLE 5
BLASTn hit against database 16S ribosomal RNA; Enterobacter soli
strain LF7 16S ribosomal RNA gene, partial sequence

| | Query | E- | % | | |
|---|---|---|---|---|---|
| | Coverage | Value | Identity | Species | Accession |
| EB-008 | 100% | 0 | 99.36% | Enterobacter soli | NR_117547.1 |
| EB-016 | 100% | 0 | 99.50% | Enterobacter soli | NR_117547.1 |
| EB-018 | 100% | 0 | 99.16% | Enterobacter soli | NR_117547.1 |

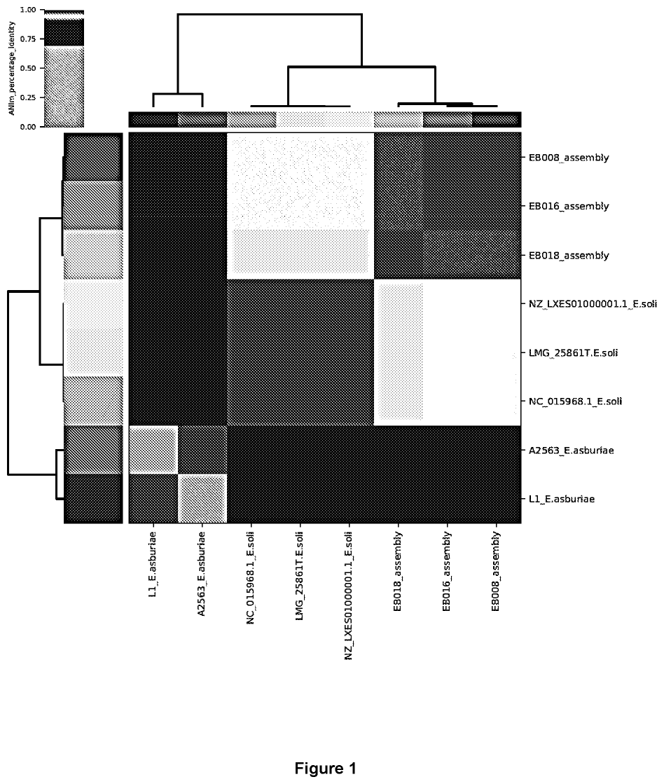

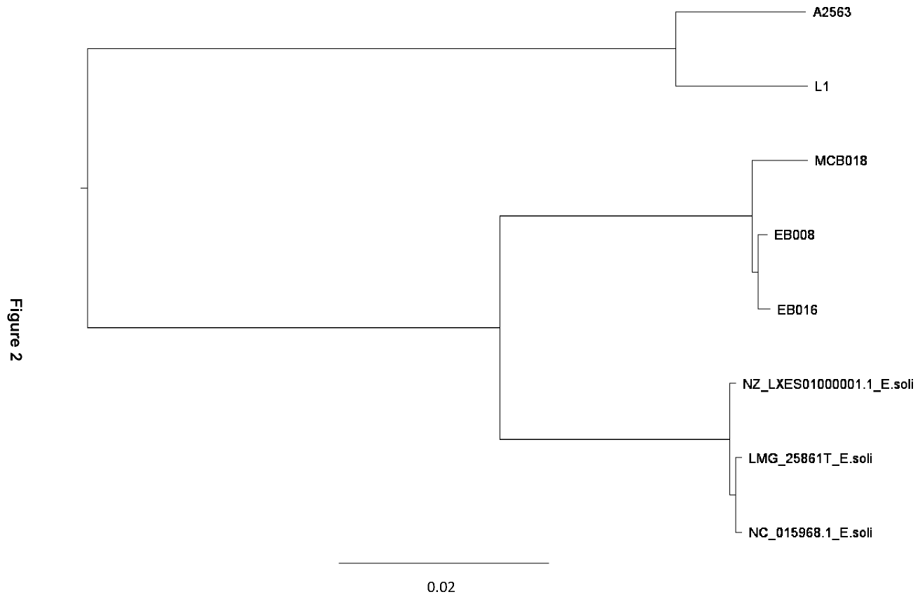

Three E. soli genome sequences and Two E. asburiae genome sequences that are publicly available on NCBI were acquired and used for average nucleotide identity (ANI) calculation (FIG. 1) and pan-genome/comparative genome sequence analysis alongside novel bacterial strains EB-008, EB-016 and EB-018 (FIG. 2). ANI values were calculated using the Pyani package (Pritchard L. 2016 https://github.com/widdowquinn/pyani) and three novel bacterial strains EB-008, EB-016 and EB-018 had >99% average nucleotide identity to each other while 94% identity to E. soli and <87% to E. asburiae isolates. Based on an ANI species cut of 95-96%, it is evident that isolates EB-008, EB-016 and EB-018 are representative of a novel Enterobacter species.

Prokka annotated novel bacterial genomes were provided to Roary (Page et al. 2015) and a total of 1327 genes that are shared by all eight strains were identified by running Roary. PRANK (Löytynoja 2014) was then used to perform a codon aware alignment and visualization of phylogenetic tree derived from core gene alignment was produced with FigTree (version 1.4.4 https://github.com/rambaut/figtree/releases). The novel bacterial strains EB-008, EB-016 and EB-018 clustered tightly together, suggesting a close phylogenetic relationship between these bacterial strains. Moreover, this cluster was separated from other Enterobacter species used in the analysis including E. soli with strong local support value (100%). This separation supports that these three bacterial strains are from a novel Enterobacter species, but closely related to E. soli.

### Example 3—Genome Sequence Features Supporting the Biofertilizer Niche of the Novel Bacterial Strains

### Nif Gene Clusters

The genome sequences of the four novel bacterial strains EB-008, EB-016 and EB-018 were assessed for the presence of features associated with biofertilization. The annotated genome sequences were assessed for the presence of the nif gene cluster (nifA, nifB, nifD, nifF, nifH, nifJ, nifK, nifL, nifS, nifW) by aligning the genome sequences against nif genes using BLASTn. The nif gene cluster was identified in all three genomes (Table 6, SEQ ID NOs: 4-16).

TABLE 6
nif genes identified within Enterobacter
strains EB-008, EB-016 and EB-018

| | | Nucleic acid |
|---|---|---|
| Gene | Enterobacter strain | SEQ ID NO: |
| nifA | EB008, EB016 | 4 |
| nifA | EB018 | 5 |
| nifB | EB008, EB016, EB018 | 6 |
| nifD | EB008, EB016, EB018 | 7 |
| nifF | EB008, EB016, EB018 | 8 |
| nifH | EB008, EB016 | 9 |
| nifH | EB018 | 10 |
| nifJ | EB008, EB016 | 11 |
| nifJ | EB018 | 12 |
| nifK | EB008, EB016, EB018 | 13 |
| nifL | EB008, EB016, EB018 | 14 |
| nifS | EB008, EB016, EB018 | 15 |
| nifW | EB008, EB016, EB018 | 16 |

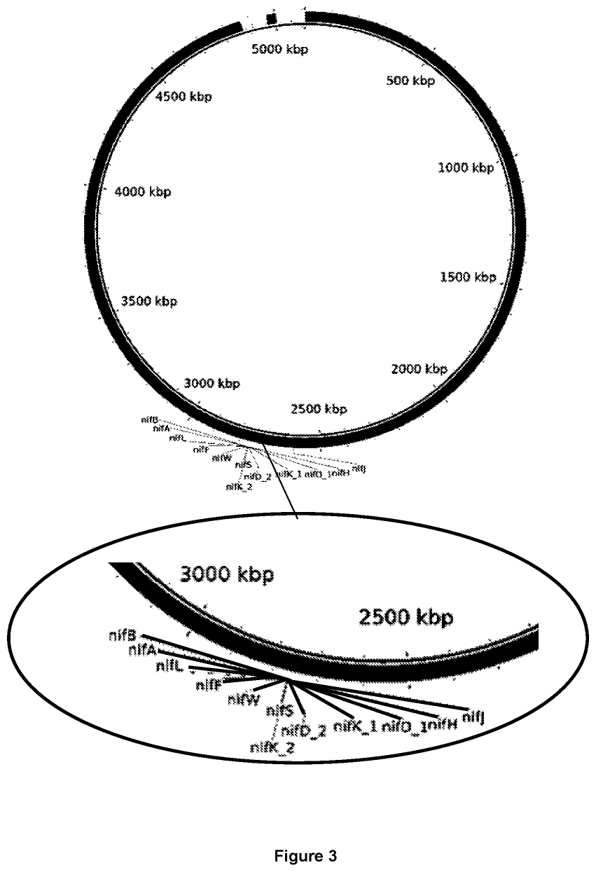

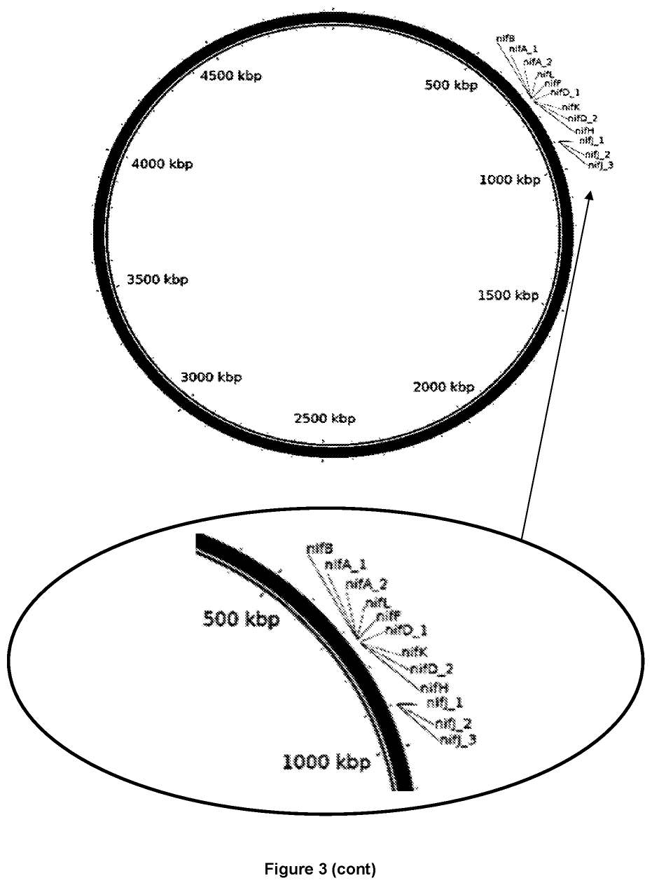

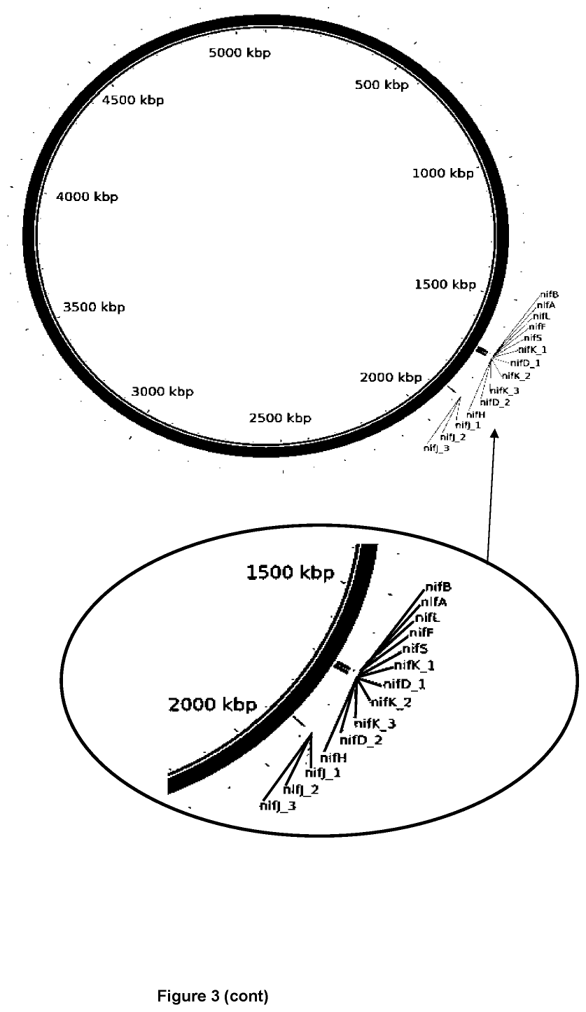

Annotated genome sequences were passed through BLAST Ring Image Generator (BRIG) (Alikhan, Petty, et al., 2011) and presence of nif gene cluster in all three genomes were graphically interpreted, and found to be localised in different regions throughout the genomes. (FIG. 3).

### Example 4—Transcriptomics Supporting the Biofertilizer Niche of the Novel Bacterial Strains

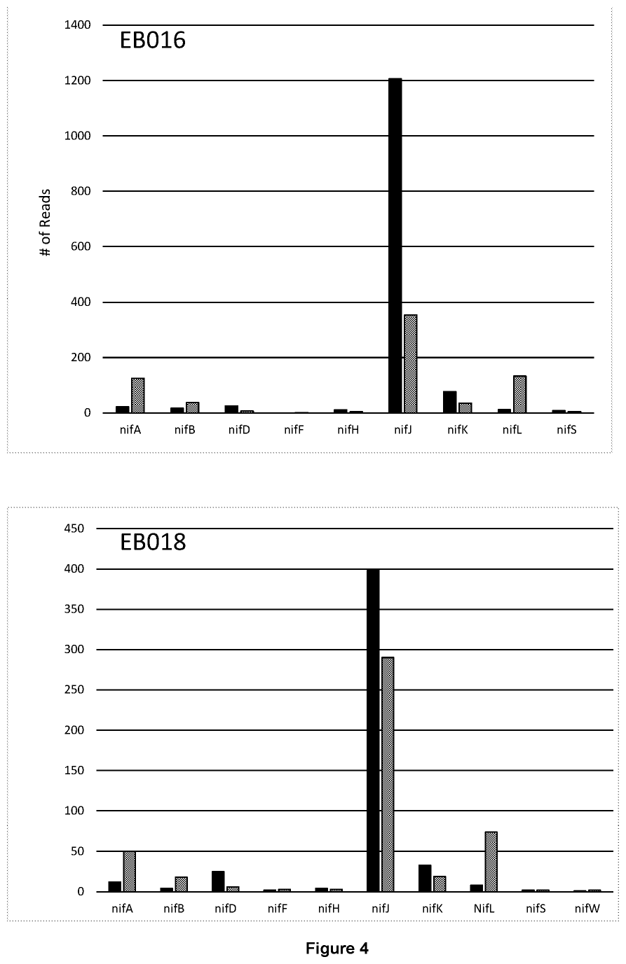

The transcriptomes of novel bacterial strains EB-016 and EB-018 were sequenced under normal nitrogen and no nitrogen conditions. The novel bacterial strains were conditioned for 24 hours on nitrogen-free Burks solid medium (Wilson & Knight 1952) and then transferred into either Burks liquid broth or Murashige and Skoog (MS) broth for 24 hours. Bacterial cell pellets from these cultures were used for total RNA extraction using the TRIzol plus RNA purification Kit (cat No: 12183555, Invitrogen). Subsequently, ribosomal RNA was removed using NEBNext rRNA depletion kit (NEB #E6310, BioLabs New England) and cDNA sequencing libraries were generated for Illumina sequencing using the NEBNext ultra-RNA library prep kit for Illumina (NEB #E7530, BioLabs New England) protocol. All libraries were sequenced using an Illumina MiSeq platform. The raw RNA-Seq reads were filtered to remove any adapter and index sequences as well as low quality bases using fastp (Chen, Zhou, et al., 2018). Reads were mapped to the nif genes of the respective isolate using the Gydle software suite (https://www.gydle.com) to obtain the abundance of reads per gene. The nif gene cluster was transcriptionally active in novel bacterial strains EB-016 and EB-018 with reads detected in nif genes of both strains (FIG. 4). NifJ (electron donor to the nitrogenase) had the highest number reads detected, followed by nifL (negative regulator) and nifA (positive regulator). Comparing the two conditions (no N and normal N), both strains responded similarly with the upregulation of nifA (SEQ ID NOs: 4 and 5), nifB (SEQ ID NO: 6), nifF (SEQ ID NO: 8) and nifL (SEQ ID NO: 14), and the down regulation of nifD (SEQ ID NO: 7), nifH (SEQ ID NOs: 9 and 10), nifJ(SEQ ID NOs: 11 and 12) and nifK(SEQ ID NO: 13) under no nitrogen.

### Example 5—Biofertiliser Activity (in Planta) of the Enterobacter sp. Novel Bacterial Strains

To assess direct interactions between the novel bacterial strains and plants, an early seedling growth assay was established in tomato. A total of 4 bacterial strains (EB-008, EB-016, EB-018 and Eschericia coli) were cultured in Burks media overnight. The following day seeds of tomato were surface-sterilised (3% bleach), washed 5 times in sterile distilled water. The seeds were then soaked in the overnight cultures for 4 hours in a shaking incubator. For control seedlings, seeds were soaked in Burks media without bacteria for 4 hours in a shaking incubator. The seeds were transferred to petri plates containing sterile filter paper, the seeds were sprayed with sterile MQ water and allowed to germinate for 7 days. Germinated seedlings were transferred to semi-solid Burk's medium (no nitrogen) and MS medium (normal nitrogen) after and the seedlings allowed to grow for 10 days. The lengths of roots and shoots were measured.

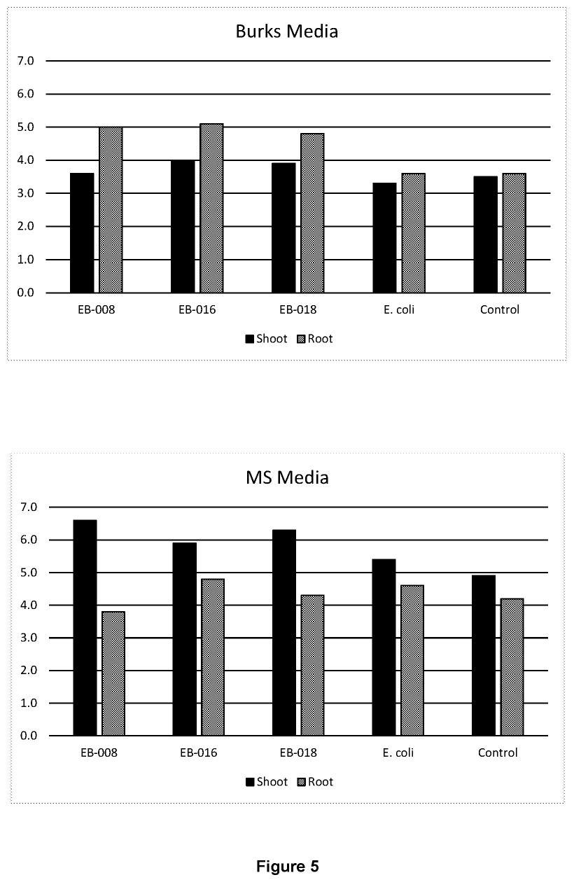

Seedlings inoculated with the novel bacterial strains were healthy with no disease symptoms recorded on leaves or roots. The length of the shoots inoculated with the novel bacterial strains were 17.0-25.8% longer than the control under normal nitrogen, and 2.8-12.5% under no nitrogen (FIG. 5). The length of the roots inoculated with the novel bacterial strains were 2.4-10.4% longer than the control under normal nitrogen, and 25.0-29.5% under no nitrogen. Statistical analysis was performed using OriginPro. The three novel bacterial strains EB-008, EB-016, and EB-018 resulted in significantly longer roots in low nitrogen media, compared to the control (p=0.05).

### Example 6—Distribution of Novel Bacterial Strains in Medicinal Cannabis Plants

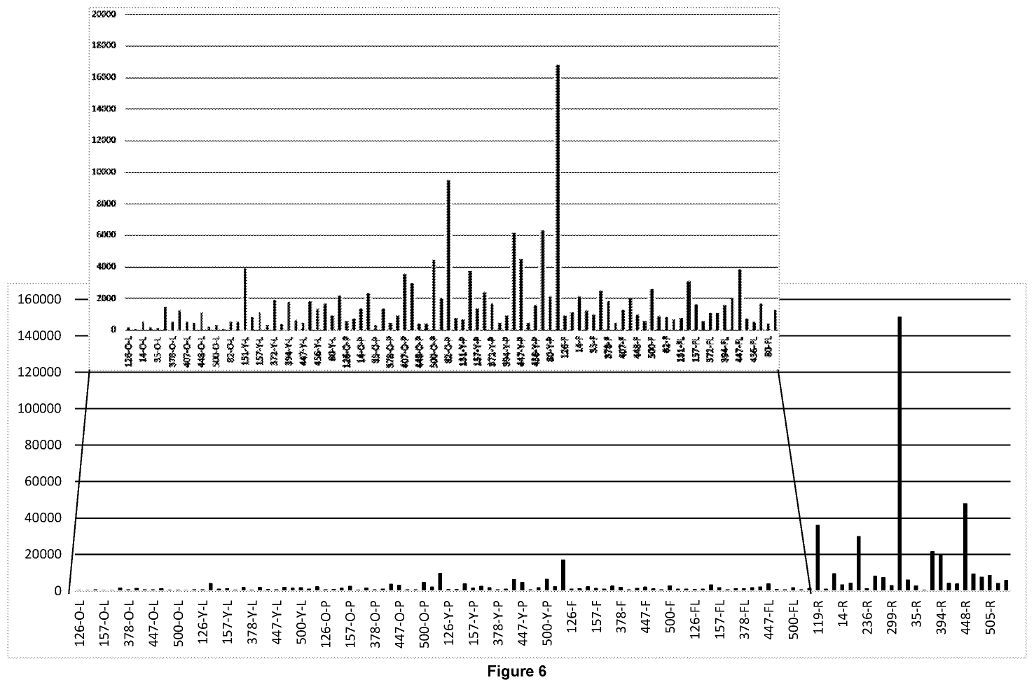

For microbiome profiling, flowers, flower bracts, leaves (old and young), petioles (old and young), roots and stem were collected from mature plants. DNA extraction was performed in 96-well plates using the QIAGEN MagAttract 96 DNA Plant Core Kit according to manufacturers' instructions with minor modifications for use with a Biomek FX liquid handling station. The bacterial microbiome was profiled targeting the V4 region (515F and 806R) of the 16S rRNA gene (SEQ ID NOs: 1, 2 and 3) according to the Illumina 16S Metagenomic Sequencing Library Preparation protocol, with minor modifications to include the use of PNA PCR blockers to reduce amplification of 16S rRNA genes sequences derived from the plant chloroplast genome and mitochondrial genome (Wagner et al., 2016). Paired-end sequencing was performed on a MiSeq to generate 2×300 bp reads. Sequence data was trimmed and merged using PandaSEQ (removal of low quality reads, 8 bp overlap of read 1 and read 2, removal of primers, final merged read length of 253 bp) (Massela et al., 2012). The Gydle software suite was used for dereplication, taxonomical assignment and removal of organelle OTUs. Reads were mapped (Gydle) to the 16S sequence of EB-016 as a representative of the four novel bacterial strains to determine the distribution of the strains through medicinal cannabis plants. Reads were identified in all organs, with numbers ranging around 1500 for flowers, flower bracts, leaves (old and young), petioles (old and young) and stems, while numbers were higher in roots (up to 150,105) (FIG. 6). As such, the novel bacterial strains appeared ubiquitously distributed across the medicinal cannabis plant, but more concentrated in roots than other organs.

Finally, it is to be understood that various alterations, modifications and/or additions may be made without departing from the spirit of the present invention as outlined herein. In particular, the present invention may be applied to a range of agricultural hosts, not limited to the Poaceae species (such as sugarcane, rice, corn) and Legumes (such as pigeon pea), as well as other horticultural species (such as sweet potato, grapes and tomato).

### REFERENCES

1. Alikhan, N.-F., N. K. Petty, N. L. Ben Zakour and S. A. Beatson. 2011. BLAST Ring Image Generator (BRIG): simple prokaryote genome comparisons. BMC Genomics 12: 402. doi:10.1186/1471-2164-12-402.
2. Chen, S., Y. Zhou, Y. Chen and J. Gu. 2018. fastp: an ultra-fast all-in-one FASTQ preprocessor. Bioinformatics 34: i884-i890. doi:10.1093/bioinformatics/bty560.
3. De Coster, W, D'Hert, S, Schultz, DT, Cruts, M & Van Broeckhoven, C 2018, ‘NanoPack: visualizing and processing long read sequencing data’, Bioinformatics.
4. Löytynoja, A 2014, ‘Phylogeny-aware alignment with PRANK’, in DJ Russell (ed.), Multiple Sequence Alignment Methods, Humana Press, Totowa, NJ, pp. 155-70, DOI 10.1007/978-1-62703-646-7_10, <https://doi.orq/10.1007/978-1-62703-646-7 10>.
5. Massela A P, Bartram A K, Truszkowski J M, Brown D G, Neufeld J D. 2012. ‘PANDAseq: PAired-eND Assembler for Illumina sequences’. BMC Bioinformatics 13(31).
6. Page, A J, Cummins, C A, Hunt, M, Wong, V K, Reuter, S, Holden, M T, Fookes, M, Falush, D, Keane, J A & Parkhill, J 2015, ‘Roary: rapid large-scale prokaryote pan genome analysis’, Bioinformatics, vol. 31, no. 22, pp. 3691-3.
7. Seemann, T 2014, ‘Prokka: rapid prokaryotic genome annotation’, Bioinformatics, vol. 30, no. 14, pp. 2068-9.
8. Vaser, R, Sovic, I, Nagarajan, N & Sikic, M 2017, ‘Fast and accurate de novo genome assembly from long uncorrected reads’, Genome Res, vol. 27, no. 5, pp. 737-46.
9. Wagner M R, Lundberg D S, Tijana G, Tringe S G, Dangl J L, Mitchell-Olds T. ‘Host genotype and age shape the leaf and root microbiomes of a wild perennial plant’. Nature Communications. 2016 Jul. 12; 7:12151.
10. Walker, B J, Abeel, T, Shea, T, Priest, M, Abouelliel, A, Sakthikumar, S, Cuomo, C A, Zeng, Q, Wortman, J, Young, S K & Earl, A M 2014, ‘Pilon: an integrated tool for comprehensive microbial variant detection and genome assembly improvement’, PLoS One, vol. 9, no. 11, p. e112963.
11. Wick, R R, Schultz, M B, Zobel, J & Holt, K E 2015, ‘Bandage: interactive visualization of de novo genome assemblies’, Bioinformatics, vol. 31, no. 20, pp. 3350-2.
12. Wick, R R, Judd, L M, Gorrie, C L & Holt, K E 2017, ‘Unicycler: Resolving bacterial genome assemblies from short and long sequencing reads’, PLOS Computational Biology, vol. 13, no. 6, p. el 005595.

1-39. (canceled)

40. A substantially purified or isolated Enterobacter sp. endophyte of a plant of the Cannabaceae family; wherein the endophyte is capable of conferring a biofertilizer phenotype to the plant or part thereof from which it is substantially purified or isolated and/or is capable of conferring a biofertilizer phenotype to a plant or part thereof to which the bacteria is inoculated.

41. An endophyte according to claim 40, wherein the biofertilizer phenotype is enhanced growth of the plant under conditions of below-normal nitrogen levels as compared to a plant that is absent of the endophyte.

42. An endophyte according to claim 40, wherein the biofertilizer phenotype is associated with expression of a nitrogen fixation (nif) gene cluster.

43. An endophyte according to claim 40, wherein one or more of the following applies:
i) the biofertilizer phenotype is associated with up-regulation of one or more of nifA, nifB, nifF and nifL;
ii) the biofertilizer phenotype is associated with down-regulation of one or more of nifD, nifH, nifJ and nifK; and
iii) the biofertilizer phenotype is associated with enhanced growth of a plant or part thereof wherein the plant part is a root or a shoot.

44. An endophyte according to claim 43, wherein enhanced growth of the root length is between about 2%-30% longer relative to an uninoculated control plant, at least 7 days after inoculation.

45. An endophyte according to claim 40, wherein the endophyte is isolated from roots of the plant.

46. An endophyte according to claim 40, wherein the plant of the Cannabaceae family is a Cannabis sativa species plant.

47. An endophyte according to claim 40, wherein the endophyte is a strain denoted EB-008, EB-016 and/or EB-018, as deposited with the National Measurement Institute of 1/153 Bertie St, Port Melbourne, Victoria 3207 Australia on 24 Nov. 2020 with accession numbers V20/025721, V20/025724, and V20/025726, respectively.

48. A plant or part thereof inoculated with one or more endophytes according to claim 40; wherein the endophyte confers a biofertilizer phenotype to the plant or part thereof.

49. A plant according to claim 48, wherein the biofertilizer phenotype is enhanced growth of the plant under conditions of below normal nitrogen levels as compared with a plant that is absent of the endophyte.

50. A plant according to claim 48, wherein the biofertilizer phenotype is associated with enhanced growth of a plant or part thereof, and wherein the plant part is a root or a shoot.

51. A plant according to claim 50, wherein enhanced growth of the root length is between about 2%-30% longer relative to an uninoculated control plant, at least 7 days after inoculation.

52. A plant according to claim 48, wherein the endophyte is inoculated into a plant or part thereof that is free of that endophyte and is stably infected with said endophyte.

53. A method for selecting a biofertilizer endophyte of a plant of the Cannabaceae family, said method comprising
a) substantially purifying or isolating one or more endophytes;
b) subjecting said one or more endophytes to microbiome profiling;
c) analysing the transcriptome of said one or more endophytes, preferably via sequencing, to identify expression of one or more genes associated with nitrogen fixation; and
d) selecting an endophyte which is capable of conferring a biofertilizer phenotype to the plant from which it is substantially purified or isolated and/or is capable of conferring a biofertilizer phenotype to a plant or part thereof to which it is inoculated.

54. A method according to claim 53, wherein the step of substantially purifying or isolating one or more endophytes includes:
a. providing one or more samples of said plant or part thereof;
b. preparing an extract(s) from said sample(s); and
c. growing bacterial colonies from said extract(s).

55. A method according to claim 53, wherein the step of subjecting the endophyte to microbiome profiling includes generating sequence data by metagenomics sequencing.

56. A method according to claim 55, wherein metagenomics sequencing is conducted using primers directed to a 16S rRNA gene and PNA PCR blockers.

57. A method according to claim 54, wherein the sample of plant material is selected from one or more ofthe group consisting offlowers, flowerbracts, leaves, petioles, roots and stem.

58. A method according to claim 53, wherein one or more of the following applies:
i) the biofertilizer phenotype is enhanced growth of the plant under conditions of below-normal nitrogen levels;
ii) the biofertilizer phenotype is associated with up-regulation of one or more of nifA, nifB, nifF and nifL; and
iii) the biofertilizer phenotype is associated with down-regulation of one or more of nifD, nifH, nifJ and nifK.

59. A method for conferring a biofertilizer phenotype to a plant or part thereof, said method including inoculating the plant or part thereof with an endophyte according to claim 40.


SEQUENCE LISTING (16 sequences)

SEQ ID NO: 1 - DNA, 1411 bp, Enterobacter sp.
ctaacacatgcaagtcgagcggtaacacagggagcttgctcctgggtgacgagcggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattaaggttaataaccttggtgattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagagggggtagaattccaggtgtagcggtgaaatgcgtagagatctggaggaataccggtggcgaaggcggcccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcggttcggccgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgct

SEQ ID NO: 2 - DNA, 1413 bp, Enterobacter sp.
agcgccctcccgaaggttaagctacctacttcttttgcaacccactcccatggtgtgacgggcggtgtgtacaaggcccgggaacgtattcaccgtagcattctgatctacgattactagcgattccgacttcatggagtcgagttgcagactccaatccggactacgacatactttatgaggtccgcttgctctcgcgaggtcgcttctctttgtatatgccattgtagcacgtgtgtagccctactcgtaagggccatgatgacttgacgtcatccccaccttcctccagtttatcactggcagtctcctttgagttcccggccgaaccgctggcaacaaaggataagggttgcgctcgttgcgggacttaacccaacatttcacaacacgagctgacgacagccatgcagcacctgtctcagagttcccgaaggcaccaatccatctctggaaagttctctggatgtcaagagtaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccactcctcaagggaacaacctccaagtcgacatcgtttacggcgtggactaccagggtatctaatcctgtttgctccccacgctttcgcacctgagcgtcagtctttgtccagggggccgccttcgccaccggtattcctccagatctctacgcatttcaccgctacacctggaattctaccccctctacaagactctagcctgccagtttcgaatgcagttcccaggttgagcccggggatttcacatccgacttgacagaccgcctgcgtgcgctttacgcccagtaattccgattaacgcttgcaccctccgtattaccgcggctgctggcacggagttagccggtgcttcttctgcgagtaacgtcaatcaccaaggttattaaccttaatgccttcctcctcgctgaaagtactttacaacccgaaggccttcttcatacacgcggcatggctgcatcaggcttgcgcccattgtgcaatattccccactgctgcctcccgtaggagtctggaccgtgtctcagttccagtgtggctggtcatcctctcagaccagctagggatcgtcgcctaggtgagccattaccccacctactagctaatcccatctgggcacatctgatggcaagaggcccgaaggtccccctctttggtcttgcgacgttatgcggtattagctaccgtttccagtagttatccccctccatcaggcagtttcccagacattactcacccgtccgccgctcgtcacccaggagcaagctccctgtgttaccgctcgacttgcatgtgttag

SEQ ID NO: 3 - DNA, 1409 bp, Enterobacter sp.
ctcccgaaggttaagctacctacttcttttgcaacccactcccatggtgtgacgggcggtgtgtacaaggcccgggaacgtattcaccgtagcattctgatctacgattactagcgattccgacttcatggagtcgagttgcagactccaatccggactacgacatactttatgaggtccgcttgctctcgcgaggtcgcttctctttgtatatgccattgtagcacgtgtgtagccctactcgtaagggccatgatgacttgacgtcatccccaccttcctccagtttatcactggcagtctcctttgagttcccggccgaaccgctggcaacaaaggataagggttgcgctcgttgcgggacttaacccaacatttcacaacacgagctgacgacagccatgcagcacctgtctcagagttcccgaaggcaccaatccatctctggaaagttctctggatgtcaagagtaggtaaggttcttcgcgttgcatcgaattaaaccacatgctccaccgcttgtgcgggcccccgtcaattcatttgagttttaaccttgcggccgtactccccgggcggtcgacttaacgcgttagctccggaagccactcctcaagggaacaacctccaagtcgacatcgtttacggcgtggactaccggggtatctaatcctgtttgctccccacgctttcgcacctgagcgtcagtctttgtccggggggccgccttcgccaccggtattcctccagatctctacgcatttcaccgctacacccggaattctacccccctctacaagactctagcctgccagtttcgaatgcagttcccgaggttgagcccggggatttcacatccgacttgacagaccgcctgcgtgcgctttacgcccagtaattccgattaacgcttgcaccctccgtattaccgcggctgctggcacggagttagccggtgcttcttctgcgagtaacgtcaatcaccaaggttattaaccttaatgccttcctcctcgctgaaagtactttacaacccgaaggccttcttcatacacgcggcatggctgcatcaggcttgcgcccattgtgcaatattccccactgctgcctcccgtaggagtctggaccgtgtctcagttccagtgtggctggtcatcctctcagaccagctagggatcgtcgcctaggtgagccattaccccacctactagctaatcccatctgggcacatctgatggcaagaggcccgaaggtccccctctttggtcttgcgacgttatgcggtattagctaccgtttccagtagttatccccctccatcaggcagtttcccagacattactcacccgtccgccgctcgtcacccgggagcaagctccctgtgttaccgctcgacttgcatgtgttag

SEQ ID NO: 4 - DNA, 1575 bp, Enterobacter sp.
atgactcagcgaacggagtcgggtacaaccgtctggcgctttgacctctcccagcagtttaccgccatgcagcgcatcagcgaggtgctaagccgggcgacggaggtcgggcagacgctacaggaagtgctgtgcgtgctgcacaacaatgcctttatgcagcacgggatgatctgtctgtacgacagccaacaagcgatccttaacattgaagccctgcaggaggccgatcagcagctcattccaggcagttcgcagattcgttaccgcccgggtgaagggttggtaggcacggtgctgtcgcaggggcaatctctggtgctgccctgtgtttccgacgaccagcgttttctcgatcgccttggattgtatgattacagcctgccgtttatcgccgtgccgctgataggaccaaactcgcagcctatcggcgtgctggcagctcagccgatggcccgctacgaggagcggctgcccgcctgcactcgttttctggaaactgtcgccaatctggtggtgcaaaccgtccgcctgatgacgccgcccagcgtcgcgccctcaccccgagcggccgccgcgcagatcgccagtcagcgtgggtgcgcaccgtcgcgatcgtttggttttgaaaacatggtcggtaaaagcgcggccatgcgtcagacgctggaaattattcgccaggtatcgcgctgggacaccaccgtgctggtgcgtggcgaaagcgggaccggtaaagagctgatagccaatgctatccaccacaattcaccgcgcgctgctgcaccgttcgtcaaattcaactgcgcggcgctacccgacacgctgctggagagtgaactcttcggtcatgaaaaaggtgcgtttaccggcgcggtacgccagcgcaaaggccgcttcgaactggcggatggaggaacgttatttcttgatgagatcggcgaaagtagcgcctcgtttcaggcgaaattgctgcgtattttgcaggaaggcgaaatggaacgcgtcggcggtgacgaaacgctgcgggtgaatgtacggatcattgccgccaccaaccgcaatctggaagaggaagtgcggcaggggaattttcgcgaagatctctactaccgcctcaatgtgatgcccatctccctgcccccgctccgcgagcgccaggaggacattgccgagctggcgcactttctggtgcgcaaaatcgcgcaaaaccagaaccgcacactgcgcatcagcgatggcgcgatccgtttgctgatgagctatagctggcccggaaacgtgcgtgaactggaaaactgccttgagcgttcggcggtgatgtcggaaagcgggctgatcgatcgcgacgtgattttgtttcatcaccgtgaaactctgccaaaaccgccacaacccagcgcgccccgcgaagagagctggcttgatcagaatctcgacgagcgacaaagattgatcgccgcgctggagaaagccggttgggtacaggcaaaagccgcccgcctgctgggaatgaccccgcgccaggtggcttaccgtattcagacgatggacatcaccatgccgcgaatgtag

SEQ ID NO: 5 - DNA, 1407 bp, Enterobacter sp.
atgatctgtctgtacgacagccaacaagcgatccttaacattgaagccctgcaggaggccgatcagcagctcattccaggcagttcgcagattcgttaccgcccgggtgaagggttggtaggcacggtgctgtcgcaggggcaatctctggtgctgccctgtgtttccgacgaccagcgttttctcgatcgccttggattgtatgattacagcctgccgtttatcgccgtgccgctgataggaccaaactcgcagcctatcggcgtgctggcagctcagccgatggcccgctacgaggagcggctgcccgcctgcactcgttttctggaaactgtcgccaatctggtggtgcaaaccgtccgcctgatgacgccgcccagcgtcgcgccctcaccccgagcggccgccgcgcagatcgccagtcagcgtgggtgcgcaccgtcgcgatcgtttggttttgaaaacatggtcggtaaaagcgcggccatgcgtcagacgctggaaattattcgccaggtatcgcgctgggacaccaccgtgctggtgcgtggcgaaagcgggaccggtaaagagctgatagccaatgctatccaccacaattcaccgcgcgctgctgcaccgttcgtcaaattcaactgcgcggcgctacccgacacgctgctggagagtgaactcttcggtcatgaaaaaggtgcgtttaccggcgcggtacgccagcgcaaaggccgcttcgaactggcggatggaggaacgttatttcttgatgagatcggcgaaagtagcgcctcgtttcaggcgaaattgctgcgtattttgcaggaaggcgaaatggaacgcgtcggcggtgacgaaacgctgcgggtgaatgtacggatcattgccgccaccaaccgcaatctggaagaggaagtgcggcaggggaattttcgcgaagatctctactaccgcctcaatgtgatgcccatctccctgcccccgctccgcgagcgccaggaggacattgccgagctggcgcactttctggtgcgcaaaatcgcgcaaaaccagaaccgcacactgcgcatcagcgatggcgcgatccgtttgctgatgagctatagctggcccggaaacgtgcgtgaactggaaaactgccttgagcgttcggcggtgatgtcggaaagcgggctgatcgatcgcgacgtgattttgtttcatcaccgtgaaactctgccaaaaccgccacaacccagcgcgccccgcgaagagagctggcttgatcagaatctcgacgagcgacaaagattgatcgccgcgctggagaaagccggttgggtacaggcaaaagccgcccgcctgctgggaatgaccccgcgccaggtggcttaccgtattcagacgatggacatcaccatgccgcgaatgtag

SEQ ID NO: 6 - DNA, 1407 bp, Enterobacter sp.
atgacttcctgcacatccgccgctggcggttcacgctgtcacaccgggcagcgtgaacctttatctgatgtcgttgccggtaaggtggcgtcgcacccctgctactcgcaaaacggccatcatcgttatgcccgtatgcatctgccggtcgcgcctgcctgtaacctgcaatgcaactattgcaaccgcaaatttgattgcagtaatgaatcgcgccccggcgtctcgtcatcgctgctgacgccggaacaggcggtagccaaagtgcgccatgtggctgcggcgatcccgcaattgtcggtggtgggcatcgccgggcctggcgacccgctggccaatattcaacgcacatttaccaccctggaaatggtgcgcgaacagctccccgacctgaaactttgcctctccaccaacgggctgatgctaccggatgcggtagagagactggtgaatgttggcgttgaccatgtcacggtgacgatcaatacgctggatgccgatatcgcggcgcaaatctacgcctggctgtggctcgacggtgaacgctacagcggacgtgaagccggtgagatcctgattgcccgccagcttgaaggcgtgcgtcgcctgacagccaaaggcgtgttggtgaagatcaactcggtattgatccccggcatcaacgatggcgttctgatcgatgtcagccgcgaactgcgtgccagcggcgcctttattcacaatattatgccgctgatcgcccgcccggagcacggtacggtgttcggtctgaacggacagccggagcctgatgcgcagatggtagcccaggtgcgtgagcgttgcggcgaggtgatgccgcagatgacgcactgccagcagtgccgcgctgacgcgatcggtatgctcggcgaagatcgcagccagcagttttccctgacgccggttgaagagttaccgcagccgtggttgccaacgctttaccaccgggcgcagttgcatgccagcattgccacgcgcggcgaatcggaagagccggatgcctgtctggtggcggttgcctccatgcgtggcgatgcgattgattgccactttggtcatgccgatcgctttagcatatacagcctgtcggcggcgggtgcggtgctggtcaacgaacgctttacgccgaaattttgtcagggaagcgacaactgcgaaccgcaggacagcgacgcgcggatggacgcggttctggatctgctggccgatgtgaaagccgtattttgcgcccgtatcggctacgggccgtggcagaaactggaagcgcgcggcattcaaccctgcgtcgatcacgcctgggagccggttaacgaggcgctgaccagctggtggcagcagtggcggaaaaaggtgcagtccgatcccgcgtcaatgggggtggcataa

SEQ ID NO: 7 - DNA, 1449 bp, Enterobacter sp.
atgagcaatgcaacaggcgaacgtaatctggagatcatccaggaagtgctggagatctttccggaaaaaacgcgcaaagaacgcagaaagcacatgatggtgagcgacccggagttggaaagcgtcgggaaatgcatcatctccaaccgtaagtcgcagcccggcgtgatgaccgtgcgcggttgctcttacgccggttctaaaggggtggtattcgggccgatcaaagatatggcccatatctcccacggcccgatcggctgcggtcagtactcccgcgccgggcggcgtaactactacaccggcgtcagcggtgttgatagcttcggtacgctcaacttcacctccgattttcaggagcgcgatatcgtgtttggcggcgataaaaagctggccaaactgattgaagagatggagacgctgttcccgctgaccaaagggatctccattcagtctgaatgcccggtcggcctgattggcgatgacattgaagccgttgccaacgccagccgcaaagccatcaataaaccggttattccggtgcgctgcgaaggttttcgcggcgtttcccagtcactcggtcaccacattgccaatgacgtgatccgcgactgggtactggataaccgtgaaggcaagccgtttgaagccggtccttatgacgtggcgatcatcggtgattacaacatcggcggtgatgcctgggcttcgcgcattttgctcgaagagatgggcctgcgcgtggtggcgcagtggtccggcgacggcacgctggttgagatggagaacacgccgttcgtcaaactcaaccttgtgcactgctaccgctcgatgaactacatctcccgccatatggaggagaaacacggtattccgtggatggagtacaacttcttcggcccgaccaaagtcgccgagtcgttgcgcaaaatcgccgacatgtttgatgacaccattcgcgccaacgctgaagcggtgatcgccaaatatcaggcgcagaacgacgccatcatcgccaaataccgtccgcgtctggaaggccgcaaagtgctgctgtatatgggcggtttacgtcctcgccatgtgattggcgcttatgaagatctggggatggaaattatcgctgcgggttatgaattcgcccataacgatgactacgaccgtaccctgccggatctgaaagaaggcaccttgctgttcgacgatgccagcagttatgaactggaagcctttgtcaaagcgctgaagccggatctgatcggctccggtattaaagagaagtacatcttccagaaaatgggcgtgccgtttcgccagatgcactcctgggattactccggcccctatcacggttatgacggctttgccatcttcgcccgcgatatggatatgacgctcaacaaccccgcgtggggccagttgactgcgccgtggctgaaatccgcctga

SEQ ID NO: 8 - DNA, 531 bp, Enterobacter sp.
atggctaacattggaatttttttcggtaccgacacgggcaaaacccgcaagattgccaaaatgatccaccaaaaactgggtgcagcggccgatgtgccggtgaatatcaaccgtaccgatctcgacacgtttctttcttatccggtcctgctgttagggacgccgacgctcggcgacgggcaactgccagggctggatgcgggctgtgaggacgcgtcatggagagagtttgtcgctcagcttgagatcaacagcctgagcggtaaaaccgtggcgctcttcggtcttggcgaccaggtgggttacccggataactttgtcagcggcctgcgcccgctgtatgacgcgctgaaaaacagcggcgcgcagattgtcggccactggccgaatgaaggctatgaattcaatgcctcatcggcgctggaagcggataaatttgtcggattagtgatcgatcaggacaatcagtatgacctgaccgacgagcgcctggatgcctggctggaaacgcttaaaccgatattgttgtaa

SEQ ID NO: 9 - DNA, 882 bp, Enterobacter sp.
atgaccatgcgtcaatgcgccatttacggcaaaggtggtatcggtaaatccaccaccacgcaaaacctggtcgccgcgctggcggagatgggcaagaaagtaatgatcgtcggctgcgacccgaaagcagactccacccgtttgatcctgcatgcgaaagcgcagaacaccattatggagatggcggctgaagtcggctccgtggaagatctggaactggaagatgtgctgcaaatcggttacggcgacgtgcgctgcgcagaatccggcggcccggaaccaggcgtaggctgtgccggtcgcggggtaattactgccatcaacttccttgaagaagaaggcgcctatgttcccgacctcgatttcgtcttttacgacgtgctgggcgacgtggtgtgcggagggttcgccatgccaattcgcgaaaacaaagcgcaggagatctacatcgtctgctccggcgaaatgatggcgatgtacgccgccaacaacatctctaaaggcatcgtgaaatacgccaaatccggcaaagtacgccttggcgggctgatctgtaactcccgtcagacagaccgtgaagatgagctgatcatggcgctggcggaaaaactcggcacccagatgatccacttcgttccgcgcgacaacattgtacagcgcgcagaaatccgccgtatgacggtgattgagtacgatccgaactgcaaccaggccaacgaataccgctcgctggcgaacaagatcgtcaacaacaccaaaatggtggtaccaacgcccatcaccatggacgaactggaagagctgttgatggaattcggcattatggatgtggaagataccagcattatcggtaaaaccgccgcagaagaaagcgcggtttga

SEQ ID NO: 10 - DNA, 882 bp, Enterobacter sp.
atgaccatgcgtcaatgcgccatttacggcaaaggtggtatcggtaaatccaccaccacgcaaaacccggtcgccgcgctggcggagatgggcaagaaagtaatgatcgtcggctgcgacccgaaagcagactccacccgtttgatcctgcatgcgaaagcgcagaacaccattatggagatggcggctgaagtcggctccgtggaagatctggaactggaagatgtgctgcaaatcggttacggcgacgtgcgctgcgcagaatccggcggcccggaaccgggcgtaggctgtgccggtcgcggggtaattactgccatcaacttccttgaagaagaaggcgcctatgttcccgacctcgatttcgtcttttacgacgtgctgggcgacgtggtgtgcggagggttcgccatgccaattcgcgaaaacaaagcgcaggagatctacatcgtctgctccggcgaaatgatggcgatgtacgccgccaacaacatctctaaaggcatcgtgaaatacgccaaatccggcaaagtacgccttggcgggctgatctgtaactcccgtcagacagaccgtgaagatgagctgatcatggcgctggcggaaaaactcggcacccagatgatccacttcgttccgcgcgacaacattgtacagcgcgcagaaatccgccgtatgacggtgattgagtacgatccgaactgcaaccgggccaacgaataccgctcgctggcgaacaagatcgtcaacaacaccaaaatggtggtaccaacgcccatcaccatggacgaactggaagagctgttgatggaattcggcattatggatgtggaagataccagcattatcggtaaaaccgccgcagaagaaagcgcggtttga

SEQ ID NO: 11 - DNA, 3525 bp, Enterobacter sp.
atgcaaactattgacggtaatggtgctgtcgcgtcagtcgcgttccgtaccagcgaagttatcgccatctacccaattacaccaagttctacgatggctgaacaggcagatgcctgggcgggaaacaggttaaaaaacgtctggggcgatgttcccagagtggtagaaatgcagtcagaagccggggccattgctgccgtgcatggcgcacttcagaccggtgctctgtctacgtcgttcacctcctcccagggcttgctgttgatgatcccgacgctgtacaagctggcaggacaactgactccctttgtattgcacgtggcggcgcgcaccgttgccacgcacgcgctctctatttttggcgatcactctgatgtcatggccgtacgacagaccggctgcgccatgctatgtgccagcagcgttcaggaagcacaggacttcgcgctgatttcccatatcgccaccctcaagagccgcgtaccgtttattcatttctttgatggtttccgcacgtcgcatgaaatcaacaaaattgttcctctggcggatgacactatcctgaacctgctgccgcaggcggagattgacgcccatcgcgcccgcgccctgaacccggaacacccggttattcgcggcacgtccgccaacccggacacctacttccagtcacgtgaagcgacgaacccatggtacaacgccgtttacgaccacgttgaacaggcgatgaacgactttgctaccgctaccgggcgtgaatacaaaccgtttgaatactatggtcacccgcaggcagagcgcgtcatcgtgctgatggggtctgcaatcggtacctgtgaagaagtggtggacgagctcctgacgcgtggcgaaaaagttggtgtgctgaaagtgcgcctttatcgcccgttcccggcaaaacacctgctctctaccctgccggaaagcacccgtagcgttgcggtgctcgaccgtaccaaagagcctggcgcacatgccgagccgctgtatctggacgtgatgaccgccctggcggaagccttcaacagcggtgaacgtgaaaccttaccgcgtgtcattggcggccggtacggtctttcctctaaagaatttggccctgactgcgtgttcgcggtgttcaatgaactggctgaagcgaagccaaaaccgcgctttacggtcggcatttatgatgacgttactaacctttcgctggtgttaccggaaaacacgctgccatcaaaagcgaagcttgaagcgctgttctatggcttaggcagtgacggtagcgtctcggcaaccaagaacaatattaagatcatcggtaactcgacgccgtggtacgcgcagggttatttcgtgtatgactcgaaaaaagccggtgggctgaccgtttctcacctgcgcgtcagcgaacacccgattcgttcggcgtacctggtgtcgcaggcagattttgtcggctgccatcagttgcagtttatcgataaataccagatggccgaacgcctcaaacctggcggtattttcctgctcaatacgccgtacagcgcagatgaagtctggtcgcgcctgccgcaggaagtgcaggcggttcttaatcagaaaaaggcccgcttctttgtagtcaacgcggcaaaaattgcgcgtgagtgcgggcttgcagcgcgtattaataccgtcatgcagatggccttcttccacctgaccaacatcctgccaggcgacagcgcgctggcggagttgcagggtgcgattgccaaaagctacagcagcaaaggccaggagctggttgaacgtaactggcaggcgctggcgctggcacgcgaatcattgttcgaagtaccgttacagcaggtgaacgccgccagcccgaaccggccgccagtagtgtctgacgctgcccctgactttgttaaaaccgttaccgctgcgatgctggcaggtctgggtgatgcccttccagtttcggcgctgccgccagacggaacctggccgatgggcaccacgcgctgggaaaaacgcaacatcgccgaagagatccccatctggaaagaagagctgtgtactcagtgtaaccactgcgttgctgcctgcccgcattctgccattcgcgccaaagtggtgcctccggaagcgatggaaaatgcaccagccagcctgcattctctggatgtaaaatcccgcgatatgcgcggacagaaatatgttctccaggtggcaccggaagattgcaccggctgtaacctgtgcgtggaagtttgcccggcgaaagatcgtcagaacccggaaattaaagccatcaatatgatgtcgcgtctggagcacgtggaagaagagaaagtgaattatgatttcttcctggacctgccagaaatcgatcgcagcacactggaacgtatcgacatccgtacttcgcagctgattaccccgctgttcgagtactccggcgcctgctctggctgcggtgaaaccccgtacatcaaactgctgacccagttgtatggtgatcgcatgttgattgccaatgccactggctgttcatctatttacgggggcaacctgccatcaacgccgtacaccacggatgccaacgggcgcggcccggcatgggcgaactccctgtttgaagacaacgcagagttcggcctgggcttccgtctgacggtcgatcagcatcgtgcccgcgtgatgcgccttctggaacagtttgccgataagatcccggcggagctgaacgatgccctgcatgctgacgcgacgccagacgtgcgccgcgagcaggttgcagcactgcgcagtgcactccagggcgttgacggtgcagaacagttactgactgatgccgatgcgctggttgagaaatctatctggctgattggtggtgacggctgggcttacgatattggatttggcggcctggatcatgtgctgagtctgactgaaaacgtcaatattctggtgctggatacacagtgctattccaacaccggtggacaggcctctaaagccactccactgggtgcggtcacgaagtttggcgaacacgggaaacgcaaagcgcgtaaagatttgggcgtcagcatgatgatgtatggccatgtctatgtcgcccagatttcactgggggcacagcttaaccagacggtgaaagccattcaggaagcggaggcctatccaggcccgtcgttgattatcgcctacagcccttgtgaagagcatggttacgatctggcgctcagccacgatcagatgcgccagctcacagccaccgggttctggccactgtatcgcttcgatccacgtcgggctgatgaaggcaaattgcctctggcgctggattcgcgtccgccgtcagatgcgctggccgagacactaatgcaggagcagcgtttccgtcgcctgaatgcgcagcagcctgaagtggcagagcagctctggaaagatgcggccgtcgacttgcaaaaacgctatgacttcctggcccagatggcaggtaaagcggaaaaatctaccaacgactaa

SEQ ID NO: 12 - DNA, 3513 bp, Enterobacter sp.
atgtccggaaagatgaaaacaatggatggcaacgcggcagcggcatggatttcgtatgcctttaccgatgtcgccgcgatttaccccattaccccctcaacgccgatggcggaaaacgtcgatcagtgggcggctgacggcaagaaaaacctgtttggccagccggtgcgtttaatggagatgcagtcagaagcgggcgcggccggtgccgtacacggcgcgttgcaggccggggcgctgaccaccacctataccgcttcgcaggggttgttgttaatgatccccaatctgtacaaaatcgccggtgaactgctgcccggtgtgtttcacgtcagcgcccgcgcgctggcaaccaactcgctgaacattttcggcgatcatcaggatgtgatggcggttcgccagagcggctgcgcgatgctggcggagagcaacgttcagcaggtgatggatctctcggcggtagcacacctctccgccattaaaggccgcgtaccttttattaacttctttgatggctttcgtacttctcacgagatccagaaaattgaagtactggagtatgacgatctggcgccgttgctggataccgacgcgctgaaccgctttcgccgtaacgccctgaacccggatcatccggtgattcgcggcaccgcgcaaaaccccgatatctattttcaggagcgggaagctgccaaccgctactacgatgcgctgccggaaatcgtcgaaaattatatggcggacatctatcgcctgaccggtcgcgaataccacctgtttgattactacggcgcagacgatgctgaacagatcattgtggcgatggggtcggtgtgcgacaccatttccgaagtggtggatgcgctaatcgacagtggtgaaaaagttggcctggtgacggtgcatctgttccggcctttttcgctgtcgcacttcttcgccaaaatcccgaaaagcgttaaacgtattgcggtgctcgatcgcaccaaagagccgggctcgcaggcagaaccgctgtgcctggatgtgaaaaatgcgttttatcaccagcatgacgcgccgctgattgtcggtgggcgctatgcgctgggcggtaaagatgtgctgccggggcatattgtctcggtgtttgagaacttgaaaaaaccgcttccgaaagatggttttacggtggggattttcgacgatgtcacgcacacctcgctgccggtgccttctcatgaggttcacgtttcccgcgaggggatcaccgcctgtaaattctgggggctgggttccgacggtacggtcagcgctaacaagagcgccatcaagatcatcggtgataaaacgccgatgtatgcgcaggcctactttgcctacgattcgaaaaaatccggcggcattaccgtttcgcacctgcgttttggtgaacggccgattacctcgccatacctgatccaccgcgcggattttatcgcatgctcgcagcaatcctacgtcgacaagtacgatctgctggaggggctgaactctggtggtacattcctgctgaactgcacctggtttggtgaagagctggaagcgcggctgccaaacagtatgaagcgttatatcgcccgccaggggatccgtttttacaccctcaatgcggtggaaattgcccgtaagctggggctgggcgggcgctttaacatgctgatgcagtcggcattcttcaaactggcggggattattgaaccgcaagcggcatccgattacctgaagcaggcagtcgagaaatcctacggcagtaaagggcaaaaagtggtggatatgaacaatgccgccattgatttggggatggaagccatccaggaagtgatggtgccggaacgctgggcgacgctggaagatgacgcggtggaagatacgcggatgatgccggattttatccgcgacattctgcaaccgatgaaccggcagaacggcgacaaactgccagtcagcgcctttgtcggtatggaggacgggacattcccgacgggaaccgcggcgtgggagaaacgcggcattgctttgcaggtaccggtgtggaaccccgaaggctgtacccagtgcaatcagtgcgcctttatctgcccgcatgctgccgttcgtccggcactgctgaatcaggaagagcgcgatgtggcaccggtagcgttgttgagtaaagcggcgcagggggcgaaggattacgactatcatctggccatttcgccgctcgattgttccggctgcggcaactgtgtcgatatctgcccggcgaaggggaaagcccttacgatgcagccgctggagagtcagttagcgatggaaccggtgtgggactatgcgctggcgctgtcgccaaaaagcaatccgttcaacaaaaccacggttaaaggcagccagtttgaaaccccactgctggagttttcaggtgcctgtgcgggctgcggtgaaaccccgtatgcgcggctggtgacgcagttgttcggcgatcgcatgatgatcgcgaatgccacgggttgttcgtctatctggggggccagcgcgccgtcaattccgtggaccaccaaccataaaggacaaggcccggcatgggctaactcgttgtttgaagataacgccgagtttggtctggggatgatgctcggcgggcgcgcggtacgtgaacagctcgccagtgatgcgatggatgcactggctgcgccgcatagcgaggcgttacagcaggcggtgcagcaatggctggaaagcaaagatcgaggtgaaggcacgcgcgagcgcggcgagcaactgagcgcgttgctggcgcaggagaagggcgacgatccgctgctgaaccgcatgtaccagaaccgcgactatttcgctaaacgttcgcagtggatttttggcggcgacggttgggcgtatgacatcggttttggcggcctcgatcatgtgctggcttccggcgaggatgtgaatatcctggtgtttgatactgaggtgtactctaacactggcggtcagtcgtcgaaatcgacgccggttgcggcaattgccaagtttgccgccgaaggcaaacggacgcgcaagaaggatctcggcatgatggcggtgagctacggcaatgtgtatgtcgcgcaggtggcaatgggcgcggataaagcccagacgctgcgggcgattgctgaagcggaagcgtggcctggcccgtcgctggtgatcgcttatgcggcctgtatcaaccacggcctgaaagccgggatgggatgcagtatgcgcgaagcaaaacgcgcggtggactcgggctactggcatctgtggcgctataacccgcagctgatcgcgaagggtaagaatccgtttattctcgactccgacgaaccagaggagagcttccgcgacttcctgatgggtgaagtgcgctacgcagcgctgggccgcactgcaccggaactggcggatcaactcttcgagcaaaccgagcaggacgcgaaacagcgtttagcgcagtatcagcgaatggcgggggagtaa

SEQ ID NO: 13 - DNA, 1563 bp, Enterobacter sp.
atgagccaaagtgttgagaaaattcaaaactgtcatccgctgtttgaacaggatgcgtaccagatgctgtttaaagataaacggcaactggaagaggcccacgatccggcgcgcgtgcaggaggtctttcaatggaccaccacggcagagtatgaagcgcttaactttcaacgcgaagcgctgactatcgatccggcaaaagcctgccagcctctgggcgcggtactttgcgcgctgggctttgccaataccctgccgtatgttcacggctcccaggggtgcgtggcctatttccgcacctattttaaccgtcactttaaagagccgattgcctgtgtttccgactcgatgacggaagatgcggcagtattcggcggcaacaacaacctgaacaccgggttgcagaacgccagcgccctctacaagccggaaatcattgccgtctccaccacctgtatggcggaggtcatcggcgacgacctgcaggcgtttattgctaacgccaaaaaagacggctttatcgacgcggcgatcccggtgccttacgcgcatacgccgagctttatcggcagccatatcaccggctgggacaatatgtttgagggcttcgcccgtacctttaccgccgattacagcgggcagccgggcaagctaccgcgtatcaatctggttagcggatttgaaacctatctcggcaatttccgcgtgctgagacgcatgatggagcaaatggaagtgccgtgcagtctgctttccgatccctccgaagtactggataccccggctgacgggcattaccagatgtatgccggcggtacgacccagcaggagatacgcgaagcgcctgacgctatcgacaccctgctactgcaaccctggcagctggcgaaaactaaaaaagtggtgcaggaaagctggaaccagcccgccaccgaggtgcaaatcccgatggggctggccggaaccgacgaactgctgatggcagtaagccagttaaccggcaaagccattccggatagcttagcgctggaacgcggccggctggtggacatgatactcgactcccacacctggctgcacggcaagaaattcggcctgttcggcgacccggattttgtcatggggctgacccgcttcctgctggaactgggctgcgaaccgacggtgattctgtgccataacggcaacaaacgctggcagaaagcgatgaagaaaatgcttgaagcctcgccgtacggtaaagagagcgaagtctttatcaactgtgatctgtggcatttccgctcgctgatgtttacccgtcagccggactttatgatcggcaactcctacgccaagtttatccagcgcgatacgctggcgaagggtgagcagtttgaagtgccgctgatccgcctcgggttcccgctgttcgatcgccaccatctgcaccgccagaccacctggggttacgaaggggccatgaatatcctcaccacgctggttaatgcggtgctggagaaagtcgacagagagaccatcaagctcggcaaaaccgactacagcttcgatcttatccgttaa

SEQ ID NO: 14 - DNA, 1488 bp, Enterobacter sp.
atgaccctgaatatgatgatggacgccgcagcgcccgccgagatcgccggagcgctctcacaacagcatcccgggttgtttttcaccatggttgaacaggctcccgttgcgatttccctgactgatgccgatgcccacattctgtatgccaacccggcgttttgccgccagtcggggtatgagctggaagagttgttgcagcaaaacccgcgcctgctcgccagtaaacagacgccgcgtgaaatctaccaggatatgtggcacaccctgctgcaacaccgtccgtggcgcgggcaactgatcaatcgtcgccgcgacggcagcctgtttctggtggagatcgacatcaccccactgtttaatacgttcggcaaactcgaacattacctggccatgcagcgcgacatcagtaccagctacgcgctggaacaacggctgcgtaatcatatgacgctgactgaagccgttttgaataatattccggcggcggttgtagtggtggatgaacgcgatcaggtagtgatggataacctcgcctataaaactttttgcgccgattgcggcggtaaagagctgctgaccgaactcaatttttccgcccataaagcggagctggcgcagggcctggtgctgccggtagcgctgcgcggcacagtacgctggttgtccatcacctgctgggcgctgccgggcgtcagtgaggaagcgggtcgctactttattgatagcgccgtgccgcgcacgctggtggtgatcaccgacaatacccagcagcagcaacaacaggaacaggggcgtctcgaccgtctgaagcagcagataaccagcggtaaattgctggcggcgatccgcgaatcgctggacgccgcgctggtgcaactcaattgcccaattaatatgctggccgccgcccgtcgtttaaatggcgacgatcacagcaatctggcgctggatgccgcctggcgtgaaggcgaagaagcgatggcgcggctgcaacggtgccgtccgtcgctggagctggaaagcccggcagtctggccgctccagccgttccttgacgatctgctcgctctgtatcacctccgatataaccagggcgaaaacctgcaaattgagctggaatcgccctatctggtgggctttggtcagcgaacacaactgcttgcctgcctgagcctgtggctcgacagaacgctggatattgccgctgagctacgtgatttcacggtacagactcagctttacgcccgcgaagagagcggctggctgtcgttctatttaaacgacaatgtaccgctgactcaggtgcgctacacccattcccccgatgcgctcaatgcccccggtaaaggcatggagctgcggctgatccagacgctggtcgcccaccatcgcggcgcaattgaattgacctcccgccctcagggaggcacctgcctgaccctgcgtttcccgttatttcactctctgaccggaggctcactatga

SEQ ID NO: 15 - DNA, 1203 bp, Enterobacter sp.
atgaaacaggtatatctggataacaacgccaccacgcgaatcgatccaatggtgctggaagcgatgatgccctttctgaccgagcattatggcaacccctcttcgattcatgatttcggcacgccttcccgtgaggcccttgagcgcgcgcaccaacaggttgccgccctgcttggggccgatcataccagcgagattattttcacctcctgcgcaaccgaagccacgtcaaccgcgctgcacgcctgtgttgaactgatgccggagcgccgggaaattatcaccactgcagtggagcacccggccacgctcgccgtttgtgaacatctggagcgtcagggttaccttatccaccgcattggtgtgaatagcgacggtgcgctggatatggagcagtaccgccaggcactcagcacacgagtggcggtggtgagcgtgatgtgggcaaacaacgaaaccggcgtgctgtttccggtgattgaaatggcggccatggcccatgaatacggcgcgctgttccactgcgatgcggtacaggcggtcggcaaaattccgctggatattggtcgcacgcagatcgatatgctctcctgctctgcgcacaaactgcacgggccgaagggtgtcggctgcctctatctgcggcgcggaacgcgttttcgtccgctgctgcgcggcggccatcaggagcgcggacgccgcgcagggacggaaaacattgccgggattgtcggcatgggtgcagcctgcgaacttgcgcagatccatctgccaggtgcctcctcgctggcacaactgcgcgatcgactgcaggaagggcttattgcgcaggtgccttccgtgatggtaatgggcggcaaccagccgcgcgttccaggcaccaccaatctggcgtttgagtttatcgaaggggaagccattttattactgctcaaccaggtgggaattgcggcatcgagcggcagcgcctgtacctccggttcgctggagccttcgcatgtcatgcgggcgatgaacattccttacaccgccgcgcacggcagtattcgtttctcgctctcgcgctacacgcgtgagaaggagatcgactacgtgatcgacaccctgccaacgattattgcccgcctgcggatgctctcgccctactggcaggagggaaaacccggggcggctgaatcagcatttgcgccgttatacggctaa

SEQ ID NO: 16 - DNA, 258 bp, Enterobacter sp.
atggagtggttttatcaaatccccggcgtggatgagctcgataccgccgagtctttttttgaattctttagcgtgccatatgacccgctggttctgcgccactgctgtctgccggtgctgcgggaatttcaccagcggctgcggcaaaacgtaccgctgcgcaatttgcttgaagaggcgccccgcgccccgtggctactggcgcgccggttgctgacggaaagctatcagcactaccttccggagcatacctcatga